(19) United States
(12) Patent Application Publication
(10) Pub. No.: US 2011/0086799 A1
(43) Pub. Date: Apr. 14, 2011

(54) GnRH II Analogs and Uses Thereof in the Immune System

(76) Inventor: Theresa M. Siler-Khodr, San Antonio, TX (US)

(21) Appl. No.: 12/871,578

(22) Filed: Aug. 30, 2010

Related U.S. Application Data
(63) Continuation-in-part of application No. 10/915,553, filed on Aug. 10, 2004, now abandoned.
(60) Provisional application No. 60/494,259, filed on Aug. 11, 2003.

(51) Int. Cl. C07K 7/06; A61K 38/10; A61K 38/08; G01N 33/53; A61P 37/00; A61P 25/00

(52) U.S. Cl. 514/9.7; 530/328; 436/501

(57) ABSTRACT

Specially designed GnRH II analogs that are resistant to degradation by peptidases, are disclosed. The GnRH II analogs incorporate D-Arg, D-Leu, D-tBu-Ser, D-Trp, D-Asn or other active D amino acids at position 6 and aza-Gly-amide or other amino acid-amide at position 10. The D-Arg (6)—GnRH II-aza-Gly (10)-amide, D-Asn—GnRH II-aza-Gly (10)-amide, and D-Leu(6)—GnRH II-aza-Gly(10)-amide analogs are also provided, and demonstrate preferential binding to immune system GnRH receptors. These GnRH II analogs or their antibodies may be used in pharmaceutical preparations, and specifically in treatment of various immune system disorders. Antibodies to GnRH II, Applicant's GnRH TI analogs, and GnRH receptors can be used for the detection of GnRH II or the GnRH II analog or the GnRH II receptors as a diagnostic tool and/or to monitor treatment.

This is a continuation-in-part patent application claiming priority to U.S. patent application Ser. No. 10/915,553, filed on Aug. 10, 2004, which claims priority to U.S. Provisional Patent Application Ser. No. 60/494,259 filed Aug. 11, 2003.

### BACKGROUND OF THE INVENTION

1. Field of the Invention

The present invention relates generally to the immune system, and more particularly to a unique GnRH II analog designed to be useful in the immune system and with certain immune system disorders, and its use within the immune system as a protagonist or antagonist. Such disorders can include allergies, asthma, graft versus host disease, immune deficiency diseases, and autoimmune diseases, inflammatory responses, as well as immune processes regulating implantation and pregnancy and tumor rejection.

2. Description of the Related Art

Applicant has found that GnRH II analogs are useful in the immune system and in various immune system disorders. Applicant performed a number of studies on GnRH II in the immune system, relating to its localization and that of its specific receptor and its effect on B cells, monocytes, macrophages, dendritic cell and natural killer cells and immune system cells and functions.

It has been reported that GnRH II stimulates T cell adhesion and homing, but these effects were only seen after twenty-four hours. Thus, this effect appears to be secondary to a more immediate action. Based on Applicant's studies of GnRH II and its specific receptor localization and its activity on leukocytes, GnRH II and Applicant's GnRH II analogs directly affect monocytes, macrophages, B cells, dendritic cells, mast and natural killer cells directly. Other than Applicant's studies, no other studies on specifically designed stable GnRH II receptor analogs have been reported.

Applicant's studies of GnRH II, Applicant's GnRH II analog and its specific receptor have led to Applicant's proposal that GnRH II regulates cells of the immune system, including but not limited to monocyte and macrophage, B cell, dendritic cells, mast and natural killer cells differentiation and function. These GnRH II receptor-mediated events participate in the regulation of what is recognized to the human body to be foreign, whether it is sperm, embryo implantation, and endometrial implant, tumor acceptance, another self protein, tissue transplantation, tumor rejection or an infection such as a virus, such as HIV. These GnRH II receptor-mediated events form the basis of Applicant's invention described herein. Applicant envisions that the GnRH II and Applicant's analogs, and its interactions with the specific GnRH II receptor, when appropriately formulated and administered, can be used to stimulate or inhibit immune function. Applicant further envisions that Applicant's analog, and the use of antibodies to GnRH II, GnRH II receptors and Applicant's analog can be used for diagnosis of immune disorders, monitoring the treatment of immune disorders, and treating immune disorder.

### BRIEF SUMMARY OF THE INVENTION

The present invention relates to analogs of GnRH II specifically designed to be administered into the immune system and used in immune system disorders, such as allergies, asthma, graft versus host disease, immune deficiency diseases, autoimmune diseases, inflammation and tumor rejection, immune processes regulating implantation and pregnancy, endometriosis, uterine fibroids, and immune-involved diseases. Applicant's analogs are designed to be stable in blood or tissue and resistant to degradation by peptidase or other enzymes. Applicant's analogs are designed, and as discussed herein are intended to be administered directly into the immune system for binding to immune system GnRH receptors. Applicant's analogs are not designed to be mediated through the hypothalamus-pituitary-gonadal-thymus axis.

As used herein, unless otherwise specified, reference to “GnRH I” means native, naturally occurring GnRH I having the sequence set forth in SEQ ID NO: 1 in the Sequence Listing attached hereto. As used herein, unless otherwise specified, reference to “GnRH II” means native, naturally occurring GnRH II having the sequence set forth in SEQ ID NO: 2 in the Sequence Listing attached hereto. As used herein, unless otherwise specified, reference to “GnRH II analogs,” “Applicant's analogs”, “the analogs of the present invention” or the “analogs” means Applicant's GnRH II analogs having the sequence set forth in SEQ ID NO: 3 in the Sequence Listing attached hereto.

Applicant specifically incorporates the material contained in the attached Sequence Listing herein by reference. The Sequence Listing is attached in an ASCII text file, identified by the name “Sequence Listing.txt”, which was created on Aug. 27, 2010. The Sequence Listing file has a size of 1.08 KB.

Applicant's GnRH II analogs of the present invention may act either as an agonist of GnRH II with acute direct action on the immune system or as an antagonist using chronic delivery at immune system receptors leading to down regulation. Applicant's analogs may also act as a pure antagonist of immune system GnRH II at the GnRH II receptor.

The analogs of the present invention are resistant to enzymatic degradation by enzymatic activity of peptidases or other enzymes. The GnRH II analogs' resistance to degradation by peptidases or other enzymes is due to the substitution of a D-amino acid at position 6, and the substitution at the C-terminus (position 10 of Applicant's analogs) with an amino acid-amide.

Specifically, Applicant's analogs are GnRH II analogs that are modified at the C-terminus by an amino acid-amide substitution. Any suitable amino acid-amide substitution at the C-terminus, including but not limited to aza-Gly¹⁰-NH₂ substitution, may be used making the sequence more stable in the circulation and in the immune system and lymph. The substitution at the C-terminus resists degradation by post-proline peptidases present in the blood, lymph and tissues. Applicant's studies have shown that substitution at the C-terminus with an amino acid-amide makes Applicant's analogs more stable in the blood and lymph and higher in binding affinity than substitution at the C terminus with an ethylamide.

Since human pituitary, blood and lymph also contain an enzymatic activity by endopeptidases that can degrade GnRH II at the 5-6 position, the present GnRH II analogs have also been designed to inhibit the endopeptidase degradation by having substitutions in the 5-6 position of the molecule. Specifically, the GnRH II analog of the present invention is also substituted at the 6-position with a D-Arg or other D-amino acid. Any D-amino acid is suitable to substitute at position 6 to reduce endopeptidase degradation.

The substitutions of Applicant's analogs at position 6 with a D-amino acid and the C-terminus (i.e. position 10 of Applicant's analogs) with an amino acid-amide are the only two positions of Applicant's analogs where substitutions are made. Importantly, the native GnRH II backbone (i.e. the native GnRH II amino acids at positions 5, 7 and 8 of the GnRH II decapeptide) must be preserved in Applicant's analogs. The native GnRH II backbone and the substitutions at positions 6 and 10 enhance the binding of the GnRH II analogs to GnRH II receptors in the immune system. The native GnRH II backbone of the analogs binds with high affinity to GnRH II receptors in the blood, lymph, thymus, spleen, and other tissues where immune cells and immune tissues are found, while the substitutions at positions 6 and 10 resist degradation, resulting in a high potency of Applicant's analogs and hence high affinity binding to GnRH II receptors. Substitution at position 10 with an amino acid-amide also increases the potency of Applicant's analogs over substitution with an ethylamide.

In fact, the stability of the present GnRH II analogs in the presence of peptidases and immune system tissues has been examined. Replacement of the native Gly¹⁰-NH₂ with aza-Gly-NH₂ made each of the GnRH II analogs more resistant to degradation by post proline peptidases. It was found that the less active an analog is as a competitor for GnRH degradation by peptidase, the more stable that analog will be in the immune system tissues and in lymph. Thus, the existing GnRH I analogs commonly used in medicine can be degraded much more rapidly in the immune system and lymph than Applicant's GnRH II analogs.

Because of the stability and high-affinity binding of Applicant's GnRH II analogs, there are several applications of the analogs to the immune system. Applicant's GnRH II analogs may be used to stimulate or inhibit over-activity of the immune system to treat such immune disorders as allergies, asthma, graft versus host disease, immune deficiency diseases, autoimmune diseases, inflammation, tumor rejections and immune processes regulating implantation and pregnancy.

Applicant's analogs can also be used to alter either or both the innate or adaptive immune system, such as bone, lymph nodes, circulating leukocytes, thymus lymphocytes, mast cells, natural killer cells, spleen, T-cell, B-cell, and antibody production. Applicant's GnRH II analogs may be administered in pharmaceutical preparations to treat immune system disorders.

In other embodiments, the invention provides GnRH II analogs with enhanced activity within the tissues of the immune system and lymphatic system as well as the bone, thymus and spleen. This can include, but is not limited to, enhanced activity with T cells, monocytes, macrophages, dendritic, mast and natural killer cells.

In addition, a method of monitoring a course of treatment or detection and localization of immune activity using an antibody to Applicant's analogs, an antibody to GnRH II or an antibody to GnRH II receptors to bind free GnRH II, GnRH II analog and/or GnRH receptors in the blood, lymph and immune tissues and cells is provided.

In other embodiments, the invention provides a method of diagnosing immune disorders using an antibody to Applicant's analogs, an antibody to GnRH II or an antibody to GnRH II receptors to bind free GnRH II, GnRH II analog and/or GnRH receptors in the blood, lymph and immune tissues and cells.

It is envisioned that Applicant's GnRH II analogs will be administered intravenously, intra-nasally, orally, transdermally, subcutaneously, vaginally or intramuscularly. However, virtually any mode of administration may be used in the practice of the invention.

### BRIEF DESCRIPTION OF THE DRAWINGS

FIG. 1 shows localization of GnRH II in the human spleen.

FIG. 2 is a graph showing stability of Applicant's GnRH II analog in serum and plasma. GnRH II Analog is stable throughout twenty-four hour of incubation in plasma and for the seventeen hours in serum following the initial serum formation.

FIG. 3 is a graph comparing antibody response for Applicant's GnRH II analog and a normal response titre. The IL-6 response in rabbit serum is shown following booster immunization with GnRH II analog (|—|), or C-ase-1 (Δ---Δ).

FIG. 4 is a graph showing the effect of GnRH isoforms on leukocyte function. The effect of GnRH II and GnRH I on GM-CSF release from human leukocytes at 3 and 20 hours is compared.

FIG. 5A is a graph showing absorption of Applicant's GnRH II analog and circulating concentration of IL-8 where Applicant's analog was absorbed when administered vaginally to a baboon.

FIG. 5B is a graph showing absorption of Applicant's GnRH II analog and circulating concentration of IL-8 and IFNγ where Applicant's analog was absorbed when administered vaginally to a baboon.

FIG. 6A is a graph showing Applicant's GnRH II analog as an immune system antagonist, showing monocyte production of PGE after 3 hours of analog administration. Applicant's GnRH II analog induced PGE production at 10⁻⁸ and 10⁻⁷ M, but concomitant addition of GnRH II reversed the activity.

FIG. 6B is a graph showing Applicant's GnRH II analog as an immune system antagonist, showing monocyte production of TNFα after 3 hours of analog administration. Applicant's GnRH II analog induced TNFα production at 10⁻⁸ and 10⁻⁷ M, but concomitant addition of GnRH II reversed the activity.

FIG. 7 is a graph showing Applicant's GnRH II analog as an agonist, showing monocyte production of IL-10 after 24 hours incubation with an agonistic embodiment of Applicant's GnRH II analog. GM-CSF was also increased by the agonistic embodiment of Applicant's GnRH II analog.

FIG. 8 is a table showing binding affinity, normalized to GnRH II of D-Arg (6)-GnRH II-des-Gly(10)-ethylamide compared to D-Arg(6)-GnRH II-aza-Gly(10)-NH₂ in the placenta. Receptor binding affinity for D-Arg(6)-GnRH II-aza-Gly(10)-NH₂ was three times stronger than the binding affinity of ethylamide at the C-terminus.

FIG. 9A is a graph showing the inhibitory activity of D-Arg(6)-GnRH II-aza-Gly(10)-NH₂ on post proline peptidases.

FIG. 9B is a graph showing the inhibitory activity of D-Arg (6)-GnRH-des-Gly(10)-ethylamide on post proline peptidases.

FIG. 10 is a graph showing circulating levels of Applicant's analog in a monkey following vaginal delivery of the analog emulsed in a universal gel.

FIG. 11 shows localization of GnRH II receptor in immune tissues.

### DETAILED DESCRIPTION OF THE INVENTION

GnRH II is the primary form of GnRH involved in the immune system. Disorders of the immune system are affected by GnRH II action. Because native GnRH II is subject to degradation in the blood, lymph, immune tissues and other tissues where immune cells and immune tissues are found, regulation of GnRH II action is best managed using stable analogs with high specificity and GnRH II affinity. The two major degradation molecules are (a) endopeptidases, which cleave GnRH II bond between the 5-6 positions, and (b) post-proline peptidases which attack the C-terminus, or tenth position of GnRH II.

Applicant has solved the problem of degradation of GnRH II within the immune system by creating novel GnRH II analogs that are modified in the sixth and tenth positions to resist attack and digestion by endopeptidases and post-proline peptidases while increasing GnRH II receptor affinity. Different embodiments of Applicant's analogs may act either as an agonist of GnRH II with acute direct action on the immune system or as an antagonist of GnRH II.

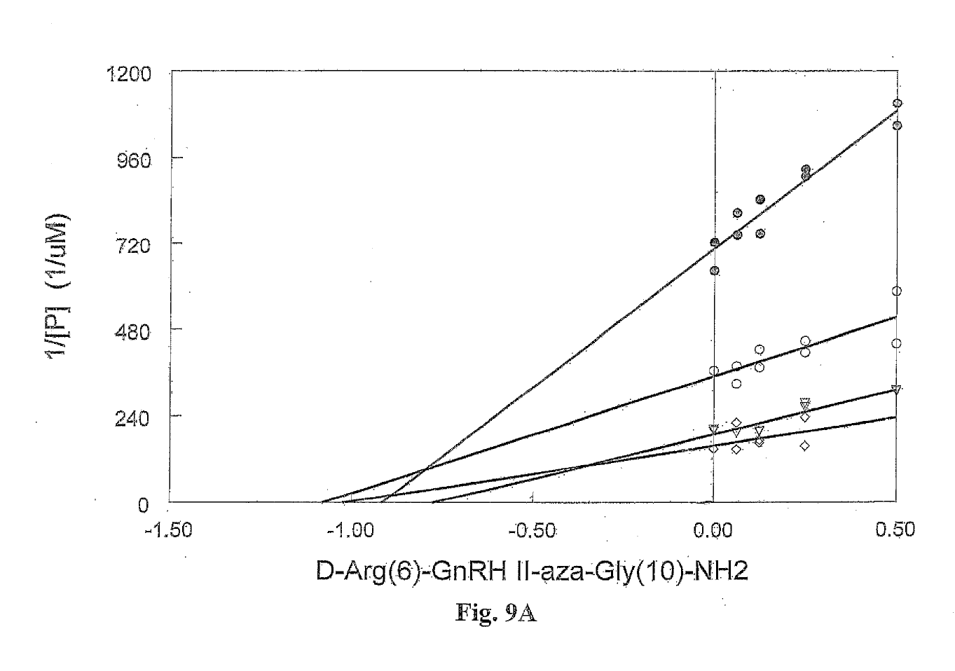

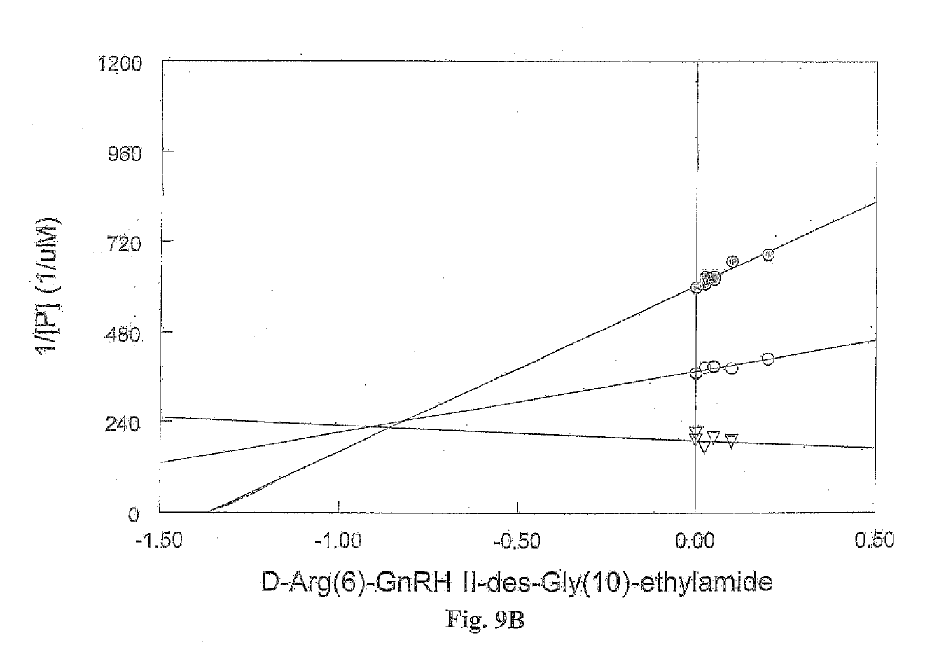

Applicant's GnRH II analogs are modified at the tenth position by an amino acid-amide substitution. Any suitable amino acid-amide substitution at the tenth position, including but not limited to aza-Gly¹⁰-NH₂ substitution, may be used making the sequence more stable in the circulation and in the immune system, immune tissues, lymph and other tissues where immune cells and immune tissues are found. Referring to FIGS. 9A and 9B, the advantage of substituting an amino acid-amide at position 10 over use of an ethylamide is shown. FIG. 9A shows the stability study of Applicant for one embodiment of Applicant's analog, D-Arg(6)-GnRH II-aza-Gly-NH₂. FIG. 9B shows the stability study of Applicant for a D-Arg(6)-GnRH II-des-Gly(10)-ethylamide. D-Arg(6)-GnRH II-aza-Gly-NH₂ is shown in FIG. 9A to be fifty percent more stable than D-Arg(6)-GnRH I-des-Gly(10)-ethylamide.

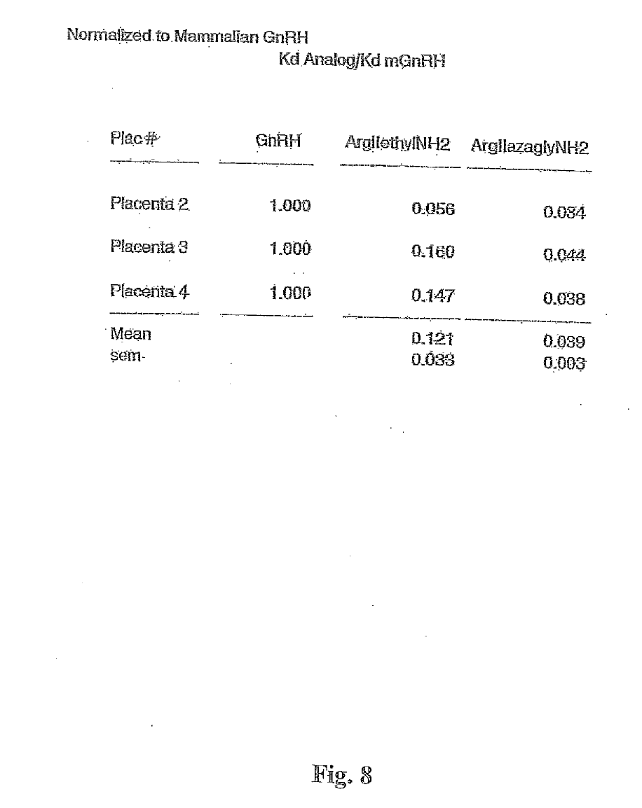

Moreover, referring to FIG. 8, the receptor binding affinity for GnRH analogs is shown. Substitution with an aza-Gly¹⁰-NH₂ shows receptor binding affinity almost three times more than an analog substituted with an ethylamide without an amino acid at position 10. Applicant's data shows that the substitution by Applicant at the tenth position with an amino acid-amide such as aza-Gly¹⁰-NH₂ resists degradation by post-proline peptidases present in the blood, lymph, immune tissues, and other tissues where immune cells and immune tissues are found, thus increasing stability therein.

The present GnRH II analogs have also been designed to inhibit endopeptidase degradation by having substitutions in the 5-6 position of the molecule. Endopeptidases in the human pituitary and blood degrade GnRH II at the 5-6 position. Applicant's GnRH II analogs substitute at the 6-position a D-Arg or other D-amino acid, including but not limited to D-Asn or D-Leu. Substitution of any D-amino acid will cause Applicant's analogs to resist degradation by endopeptidases. Any D-amino acid may be substituted at position 6 to reduce endopeptidase degradation.

However, the substitution of some D-amino acids cause Applicant's analogs to have an antagonistic affect on the human immune system, while other D-amino acid substitutions cause an agonistic affect on the immune system. In one embodiment of Applicant's analogs, Applicant substitutes D-Arg at position 6 (in addition to an amino acid-amide at position 10). Applicant has found that substitution of D-Arg and position 6 causes Applicant's analogs to have an antagonistic affect on the immune system.

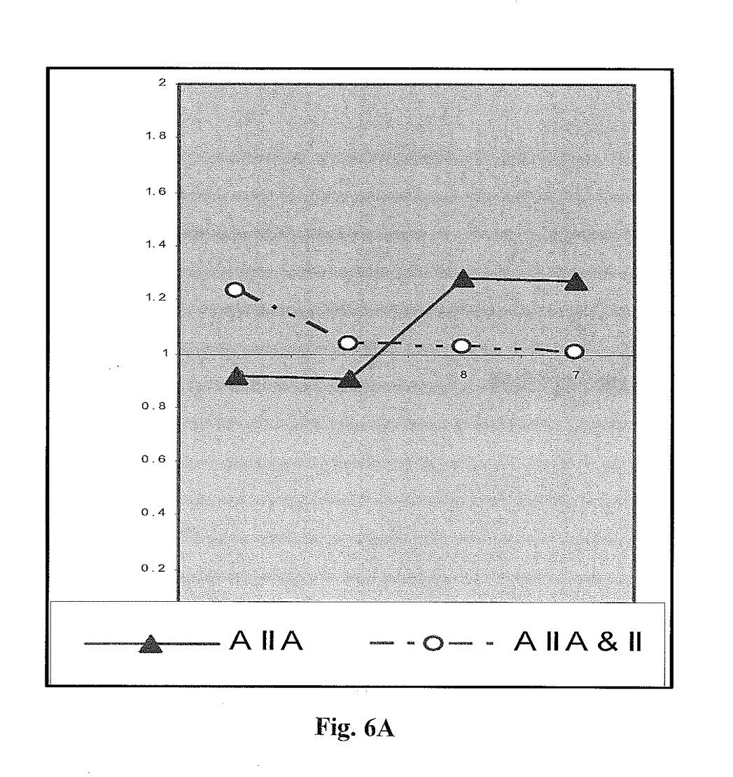

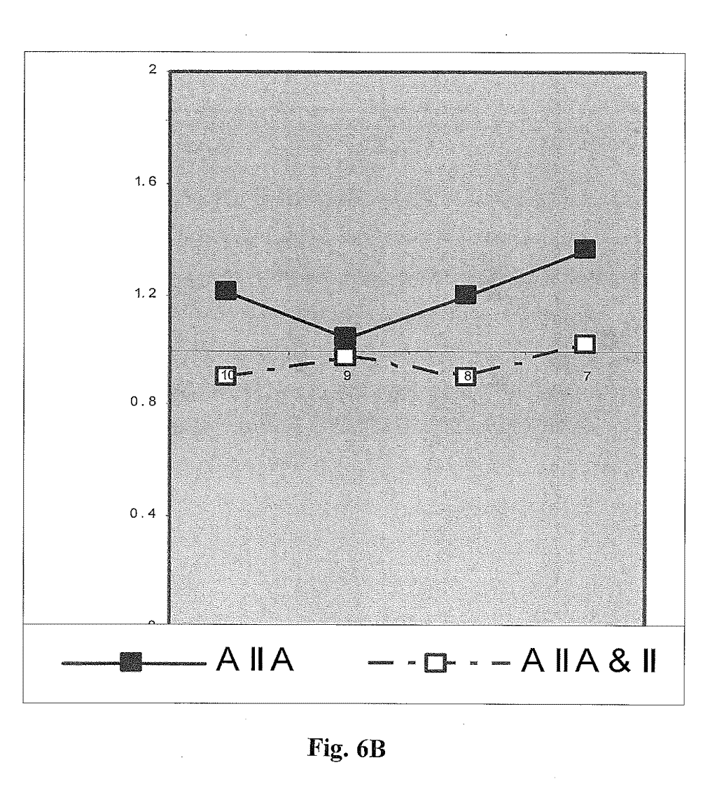

Referring to FIGS. 6A and 6B, an example of the antagonistic effect of an embodiment of Applicant's analogs is disclosed. In FIGS. 6A and 6B, “AHA” represents the embodiment of Applicant's analogs D-Arg(6)-GnRH II-aza-Gly-NH₂ Monocyte production of PGE was shown to be affected antagonistically by Applicant's analog. PGE was measured after three hours of exposure to the analog in a dose range of 10⁻¹⁰ to 10⁻⁷M (signified by the triangles in FIG. 6A), and after three hours of exposure to a combination Applicant's analog and GnRH II at a dose of 10⁻⁷M (signified by the open circles in FIG. 6A). Applicant's analog induced PGE production at 10⁻⁸ and 10⁻⁷M. However, addition of GnRH II reversed the PGE activity.

Similarly, referring to FIG. 6B, monocyte production TNFα was shown to be affected antagonistically by Applicant's analog. TNFα was measured after three hours of exposure to the analog in a dose range of 10⁻¹⁰ to 10⁻⁷M (signified by the solid squares in FIG. 6B), and after three hours of exposure to a combination Applicant's analog and GnRH II at a dose of 10⁻⁷M (signified by the open squares in FIG. 6B). Applicant's analog induced TNFα production at 10⁻⁸ and 10⁻⁷M. However, addition of GnRH II reversed the TNFα activity.

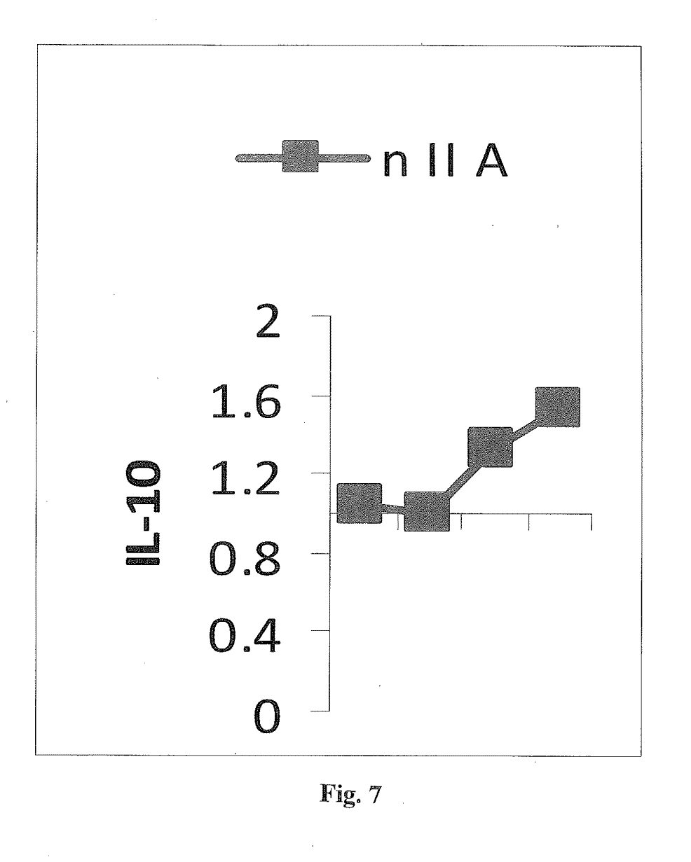

Conversely, referring to FIG. 7, monocyte production of IL-10 was shown to be affected agonistically. In FIG. 7, “nIIA” represents the embodiment of Applicant's analogs D-Asn(6)-GnRH II aza-Gly-NH₂. After twenty four hours of incubation with applicant's analog, production of IL-10 increased. GM-CSF was also increased by this analog.

The substitutions of Applicant's analogs at position 6 with a D-amino acid and the position 10 with an amino acid-amide must be made. Importantly, however, the native GnRH II backbone (i.e. the native GnRH II amino acids at positions 5, 7 and 8 of the GnRH II decapeptide) must be preserved in Applicant's analogs. The native GnRH II backbone and the substitutions at positions 6 and 10 enhance the binding of the GnRH II analogs to GnRH receptors in the immune system. The native GnRH II backbone of the analogs binds with high affinity to GnRH II receptors in the blood, lymph, immune tissues and other tissues where immune cells and immune tissues are found, while the substitutions at positions 6 and 10 resist degradation, resulting in a high stability of Applicant's analogs, while having high affinity binding to GnRH receptors. Using chimeras of GnRH I, GnRH II, or any other GnRH or GnRH like substance wherein portions of the backbone (positions 5, 7 and 8) of Applicant's analog are substituted with other corresponding amino acids from non GnRH II sources/forms of GnRH will diminish the GnRH II receptor binding capacity, and therefore are not acceptable for use as the analogs of the present invention. Said differently, to bind with high affinity directly to immune GnRH II receptors, Applicant's analog must originate from native GnRH II, and maintain the native GnRH II backbone at positions 5, 7 and 8.

Because of the stability and high-affinity binding of Applicant's GnRH II analogs, there are several applications of the analogs to the immune system. Applicant's GnRH II analogs may be used to stimulate or inhibit over-activity of the immune system to treat such immune disorders as allergies, asthma, graft versus host disease, immune deficiency diseases, autoimmune diseases, inflammation, tumor rejections and immune processes regulating implantation and pregnancy. Moreover, Applicant's analogs and antibodies of GnRH II, GnRH II receptors and GnRH II analogs can be used as a monitoring device to monitor the course of treatment of immune diseases, and to diagnose or detect expression, over expression, under expression of GnRH II receptors, or GnRH II peptide.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I

### Design of Applicant's GnRH II Analogs

The present example outlines how analogs of GnRH II with increased activity in immune system tissues are designed.

Existing GnRH I analogs are designed for activity at the pituitary GnRH receptor and with extended stability in the circulation of individuals. Yet, the existing data indicate that the immune system tissues have a high affinity GnRH receptor which differs from that in the pituitary. In addition, the degradation of GnRH I is different in the immune system. Therefore, prior known pituitary GnRH I analogs have not been designed for use at immune system sites, and potent GnRH II analogs have not been designed for use at immune system sites. The present invention provides potent GnRH II analogs for use at immune systems sites.

Applicants GnRH II analogs were specifically designed to prevent degradation of the analog in immune system tissues. This allows for the maintenance of sufficient concentrations of analog to remain active when administered to the individual and to reach the immune system tissues. Analogs GnRH II sequences that show greater affinity for the immune system receptors than for the pituitary receptor, were modified to the tenth amino acid to aza-Gly¹⁰-NH₂ analog to make them resistant to degradation in the circulation and by peptidases. The GnRH II analogs were also modified at the 6 position using D-Arg, D-Asn, or D-Leu, making them resistant to degradation by the peptidase in blood, and were modified at the 10 position making them stable in blood and the immune system tissues. These analogs have increased binding to the immune system receptors and increased metabolic stability. See FIGS. 8, 9A and 9B.

### Example II

### Localization of GnRH II in Tissues of the Immune System

Tissue of the immune system were examined for the presence of GnRH II in their cells. The presence of GnRH II demonstrated in immune cells and immune tissues of mammalian tissues that GnRH II isoforms are produced in the mammals and that they are present in the immune system.

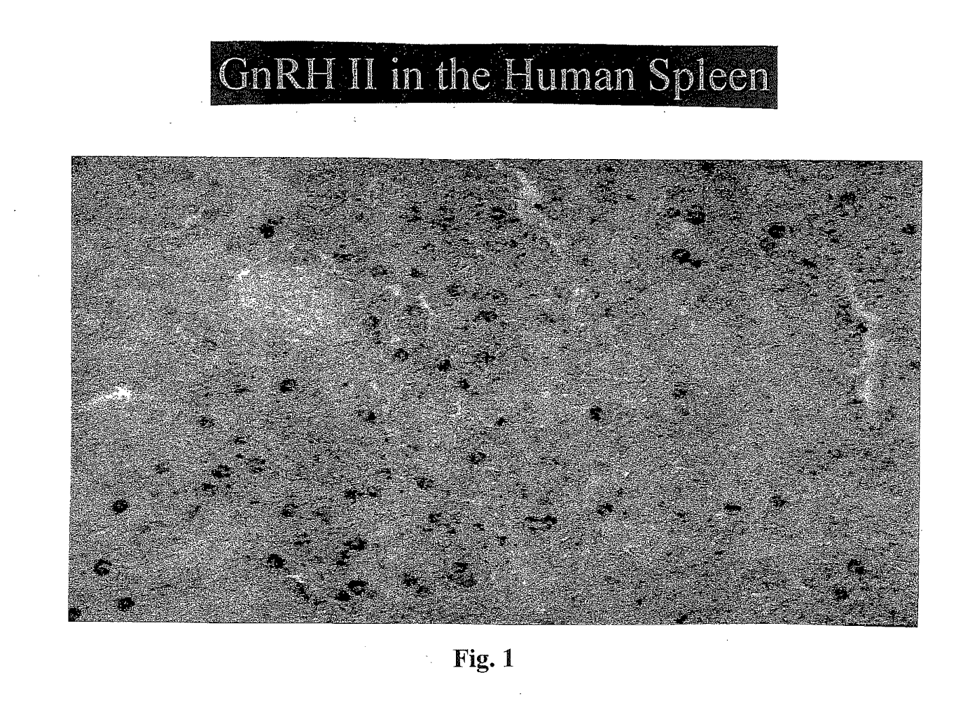

Human tissues from the thymus, spleen and lymph nodes were fixed and sectioned and plated by sections on glass slides. The human tissues on the glass slides were incubated with anti-GnRH II ( 1/100) for 1 hour at RT. The tissues were then washed with phosphate buffered saline and anti-rabbit gamma globulin conjugated with biotin is incubated for 4 minutes at 55 C. The slide was rinsed in buffer followed by blocking of the endogenous peroxidase activity. Then streptavidin horse radish peroxidase was added and incubated for 4 minutes at 55 C. Stable diaminobenzidine (5 minutes at 55 C) was used to generate the signal. The slides were rinsed, mounted and read. The presence of GnRH II was localized via the DAB using microscopy. In the immune tissues examined, spleen, thymus and lymph node GnRH II was visualized. See FIG. 1. Tissues such as atrium and liver were negative.

### Example III

### Stability Studies of GnRH Analogs

The present example demonstrated the stability of the GnRH II analogs. The enzymatic degradation of the GnRH II and its analog were studied using whole blood and plasma stability studies. A peptidase present in the immune system was used. GnRH II analogs were designed with these specific criteria in mind. The stability of these GnRH II analogs to the enzymatic activity of the peptidase and in immune system cells were examined.

The stability of most potent receptor-active GnRH II analogs in the presence of peptidase and immune system cells was identified. Each of these analogs was then studied for their ability to resist degradation over time of incubation with the immune system cells at 37° C. The reaction was stopped by freezing and the remaining GnRH I substrate, GnRH II substrate or non-mammalian analog was directly quantified by radioimmunoassay.

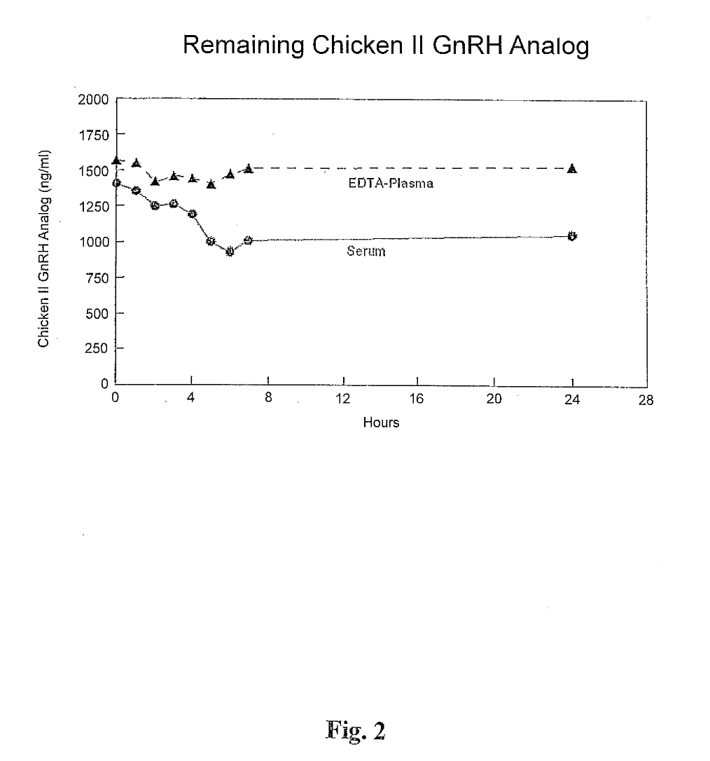

Studies using whole immune system cells were also performed. The enzymatic degradation of GnRH I was studied as described above, replacing peptidase with immune system homogenate. FIG. 2 is a graph showing stability of GnRH II analog in blood and plasma.

### Example IV

### Inhibition of Antibody Response

The production of antibodies is a function of the immune system. The ability of the immune system to respond to substances perceived as foreign by the body with the production of specific antibodies which will effect the inactivation of the substance is a function of the immune system. GnRH II or its analogs can regulate this activity in a mammal and this is a novel activity. The chronic administration of GnRH II activity can lead to the inhibition of the immune system's antibody response to a foreign substance.

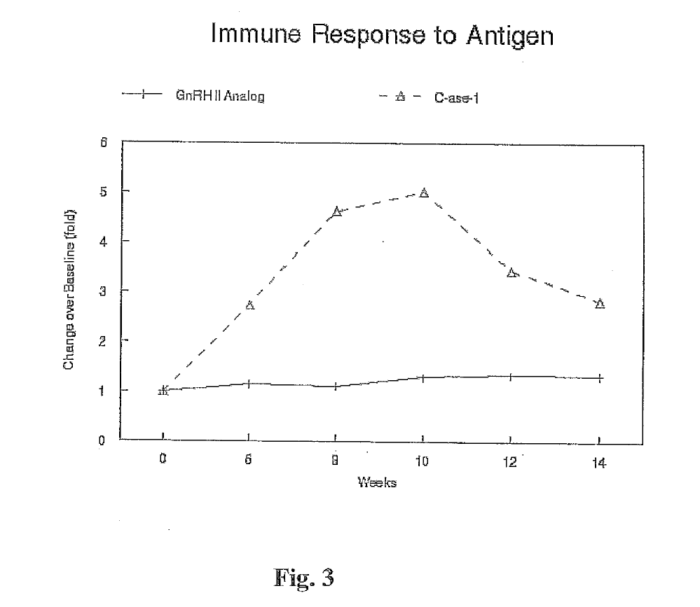

The very stable, long acting GnRH II analog, D-Arg-GnRH II-aza-Gly-amide was conjugated to KLH and injected into rabbits to generate polyclonal antibodies. The titre of the antiserum was tested for binding to D-Arg-GnRH II-aza-Gly-amide and compared to the serum before treatment in each of four animals. This was compared to the generation of anti-serum using proteins other than GnRH II or its analogs. In three animals no antibodies were detected. In one of the four animals only an antibody of low titer was generated after four treatments, which inhibition occurred with continued immunization. FIG. 3 shows the antibody response for GnRH II analog and normal response titre and IL-6 response.

### Example V

### GnRH II and Methods for Treating Immune System Disorders

The present example discloses a method by which the present invention may be used to treat immune system disorders. As a proposed dose regimen, it is anticipated that a human between 100 lbs and 150 lbs would be administered about 10 nanogram to 1.0 gram of GnRH II analogs or their natural isoforms with or without a release regulating carrier. This would be expected to be effective for treating immune system disorders when administered.

It is also anticipated that pulsatile administration will cause stimulation of its activity while chronic administration can be used to down regulate receptors leading to inhibition of GnRH II activity. In some embodiments, the dosing regimen will comprise a pulsatile administration of the GnRH II analog over a 24-hour period, wherein the daily dosage is administered in relatively equal 1/24^{th} fractions. For example, where the daily dose is about 2.4 micrograms, the patient would be administered about 0.1 micrograms per hour over a 24-hour period. Such a daily pulsatile administration would create an environment in the patient sufficient to treat certain types of immune system disorders.

### Example VI

### Use of Antibodies Specific for GnRH II for Immune System Disorders

Referring to FIG. 1, the present example demonstrates the utility for using the present invention GnRH II decapeptides to prepare antibodies that preferentially bind the GnRH II peptide sequences, or that bind the immune system GnRH II peptide or protein, Applicant's GnRH II analogs, or the GnRH receptors in the immune system. It is also anticipated that these antibodies may be used in a variety of screening assays. For example, these antibodies may be used to determine levels of GnRH II in a sample as an indicator molecule. The levels of such GnRH II may be used to monitor and follow a patient's immune system treatment. The antibodies may also be used to treat immunological disorders and diseases. The antibodies to GnRH II may be monoclonal or polyclonal antibodies.

These antibodies may be used for treatments that regulate the immune system via inhibiting the activity of GnRH II. Polyclonal antibodies may be created by standard immunization techniques, wherein the immunogen used will be native GnRH II. These peptides may be used either alone or together in a pharmaceutically acceptable adjuvant. The subject can be administered several doses of the GnRH II preparation, and the levels of the subject's antibody thereto monitored until an acceptable antibody level (titer) had been reached.

For the preparation of monoclonal antibodies, following standard techniques for the immunization of an animal, again using the peptides specific for GnRH II can be used. Once sufficiently high acceptable antibodies are reached (titer) in the animal, the spleen of the animal would be harvested, and then fused with an immortalized cell line, such as a cancer cell line, to produce a population of hybridoma cells. This hybridoma population of cells would then be screened for those that produce the highest amount of antibody that specifically bind the GnRH II. Such hybridoma cells would be selected, and then cultured. The antibody to GnRH II would then be collected from the media of the cell culture using techniques well know to those of skill in the art.

For purposes of the practice of preparing polyclonal and monoclonal antibody, the textbook Sambrook et al (1989) Molecular Cloning, A Laboratory Manual, 2^{nd} Ed., Cold Springs Harbor Laboratory, Cold Springs Harbor, N.Y., is specifically incorporated herein by reference. All of the compositions and methods disclosed and claimed herein can be made and executed without undue experimentation in light of the present disclosure.

### Example VII

### GnRH II Analogs and Methods of Use in Treatment of Conditions of the Immune System

Due to the stability of Applicant's GnRH II analogs, in the blood and lymph, the presence of binding receptors in immune system tissues, and their biological activity in immune system tissues, Applicant's analogs can be used in the treatment of conditions of or regulation of the immune system and the tissues therein. Such treatment or regulation may be for allergies or asthma, graft-versus-host disease, immunodeficiency disorders, and autoimmune disorders.

Conventional methods, known to those of ordinary skill in the art of medicine, can be used to administer the pharmaceutical formulation(s) to the patient. Typically, the pharmaceutical formulation will be administered to the patient by intramuscular injection, subdermal pellet, or nasal spray. The pharmaceutical formulation(s) can also be administered via other conventional routes (e.g., oral, subcutaneous, intrapulmonary, transmucosal, intraperitoneal, sublingual, or intrathecal routes) by using standard methods. In addition, the pharmaceutical formulations can be administered to the patient via injection depot routes of administration such as by using 1, 3, or 6-month depot injectable or biodegradable materials and methods.

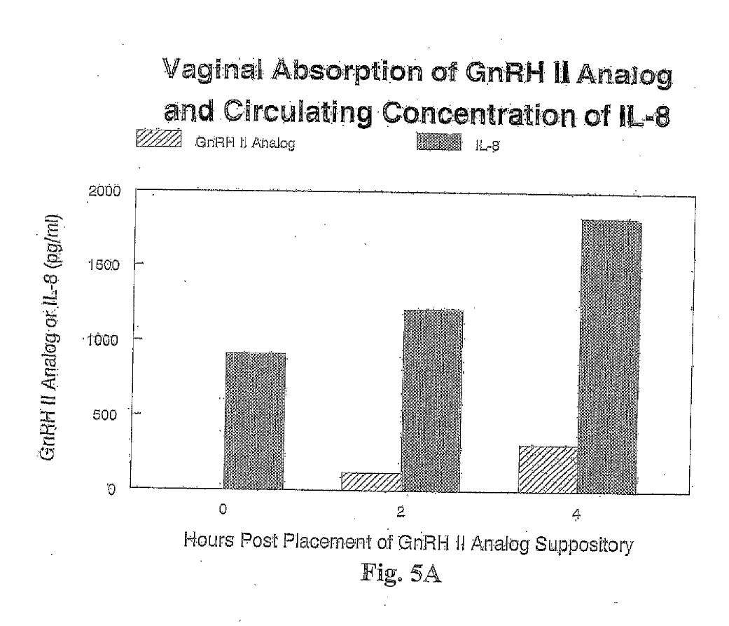

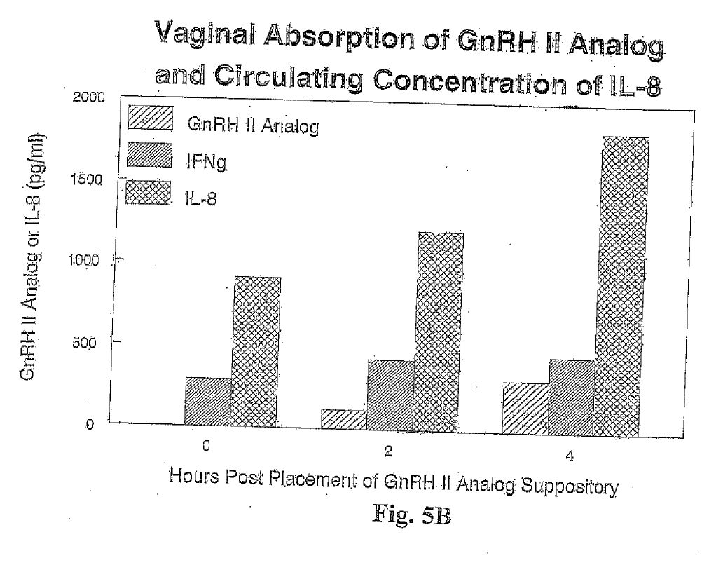

Referring to FIG. 5A and FIG. 5B, the absorption of Applicant's analog and circulating concentrations of IL-8 and IFNγ are shown. Baboons were vaginally administered Applicant's analog and measurements of GnRH II analog absorption and concentration levels of IL-8 and IFNγ were taken. Circulating concentration levels of IL-8 and IFNγ dramatically increased in response to administration of Applicant's analog.

Regardless of the route of administration, the therapeutic agent typically is administered at a daily dosage of 0.001 μg to 30 mg/kg of body weight of the patient. The pharmaceutical formulation can be administered in multiple doses per day, if desired, to achieve the total desired daily dose or as a long acting depot. The effectiveness of the method of treatment can be assessed by monitoring the patient for known signs or symptoms of the disorder.

The effectiveness of the method of treatment may also be assessed by following treatment with administration of a labeled GnRH II antibody. The antibody binding levels to either GnRH receptors or free, unbound GnRH II analog can be monitored to determine the effectiveness of the analog or its delivery.

### Example VIII

### Identification of the GnRH II Receptor in Human Immune System Tissues

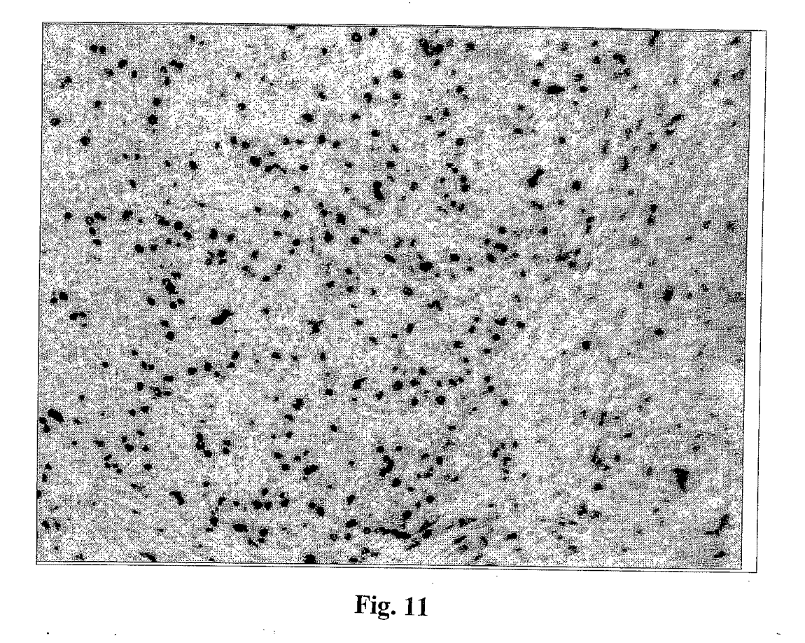

Tissues of the immune system were examined for the presence of GnRH II receptors in their cells. The presence of GnRH II receptors in the tissues of humans has not been previously described. This present investigation demonstrated in immune cells of mammalian tissues that GnRH II receptors are produced in the mammals and that they are present in the immune system. See FIG. 11.

Human tissues from the thymus, spleen and lymph nodes were fixed and sectioned and plated by sections on glass slides. The human tissues on the glass slides were incubated with anti-GnRH II ( 1/100) for 1 hour at RT. The tissues were then washed with phosphate buffered saline and anti-rabbit gamma globulin conjugated with biotin is incubated for 4 minutes at 55° C. The slide was rinsed in buffer followed by blocking of the endogenous peroxidase activity. Then streptavidin horse radish peroxidase was added and incubated for 4 minutes at 55° C. Stable diaminobenzidine (5 minutes at 55° C.) was used to generate the signal. The slides were rinsed, mounted and read. The presence of GnRH II receptor was localized via the DAB using microscopy. In the immune tissues examined, spleen, thymus and lymph node GnRH II receptor was visualized. Tissues such as atrium and liver were negative.

### Example IX

### Use of Antibodies Specific for GnRH II Receptor for Immune System Disorders

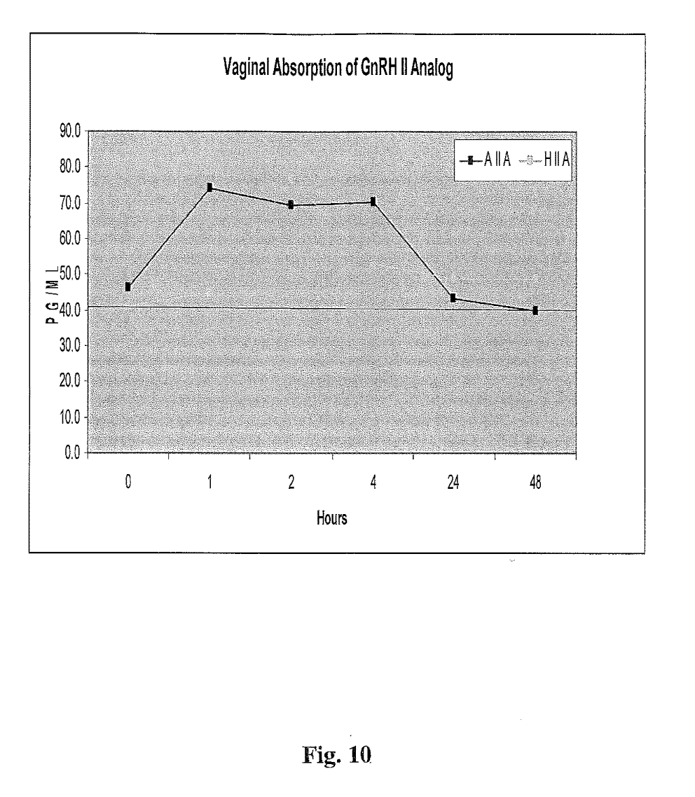

The antibodies specific for GnRH II receptor can be used to regulate immune system function. The present example demonstrates the utility for using the present invention GnRH II receptor to prepare antibodies that preferentially bind the GnRH receptor peptide sequences, or that bind the immune system GnRH receptor peptide or protein. It is anticipated that these GnRH II receptor antibodies may be used in a variety of screening assays. For example, these antibodies may be used to determine levels of GnRH II, or the GnRH receptor that binds GnRH II, in a sample as an indicator molecule. The levels of such GnRH may be used to monitor and follow a patient's immune system treatment. The antibodies to GnRH II may be monoclonal or polyclonal antibodies. Referring to FIG. 10, circulating levels of Applicant's analog (“AIIA”) following vaginal delivery in a universal gel is shown. Vaginal absorption of Applicant's analog remained in the 70 PG/ml to 80 PG/ml range up to four hours post administration.

These antibodies may be used for treatments that regulate the immune system via inhibiting the GnRH II or the activity of the GnRH II receptor. Other antiserum may interact with the GnRH II receptor to stimulate its activity. Polyclonal antibodies may be created by standard immunization techniques, wherein the immunogen used will be peptides specific to the GnRH II receptor. These peptides may be used either alone or together in a pharmaceutically acceptable adjuvant. The animal, such as a rabbit, would be administered several doses of the peptide preparation, and the levels of the animal's antibody blood levels monitored until an acceptable antibody level (titer) had been reached.

For the preparation of monoclonal antibodies, one would follow standard techniques for the immunization of an animal, again using peptides specific to the GnRH II receptor. Once sufficiently high acceptable antibodies are reached (titer) in the animal, the spleen of the animal would be harvested, and then fused with an immortalized cell line, such as a cancer cell line, to produce a population of hybridoma cells. This hybridoma population of cells would then be screened for those that produce the highest amount of antibody that specifically bind the GnRH II receptor. Such hybridoma cells would be selected, and then cultured. The antibody to GnRH II receptor would then be collected from the media of the cell culture using techniques well known to those of skill in the art.

### Example X

### Receptor Binding Activity

Referring to FIG. 8, the receptor binding activity of GnRH II and GnRH II analogs of the present invention are compared. There is a human GnRH II receptor which is distinct from the GnRH I receptor at the pituitary. Prior GnRH I analogs have been designed to increase activity at the pituitary GnRH I receptor and stability in the circulation of individuals. These GnRH I analogs do not demonstrate potent binding activity at the immune system's GnRH II receptors as they do at the pituitary's GnRH receptor. The present GnRH II analogs have been designed to interact with preference at the immune system GnRH II receptors and not the GnRH I receptor. They have also been designed to limit degradation by the immune system enzymes, present in lymphatic circulation. Binding activity of the newly synthesized GnRH II analogs has been studied in plasma. Referring to FIG. 8, the receptor binding affinity for Applicant's analogs is compared to a GnRH II analog with a substitution of ethylamide at position 10. Applicant's analog (D-Arg (6)—GnRH II-aza-Gly (10)-amide) shows receptor binding affinity almost three times more than an analog substituted with an ethylamide without an amino acid at position 10.

The newly synthesized GnRH II analogs and other commercially available analogs have been used in receptor binding studies in plasma and enzyme stability study described here. On the basis of these studies, the most receptor potent and most enzyme-stable analogs have been chosen for further biopotency studies. GnRH receptors have been purified from the fractions from immune system tissues. The purification procedure for the GnRH receptor utilized ethanol precipitation of the receptor and not the GnRH. The remaining GnRH II binding assays activity using ¹²⁵I-D-Arg-GnRH II-Aza-Gly-NH₂¹²⁵ label and GnRH II have been performed. Receptors from two different tissues from the same type of immune system cells have been used to study each of these analogs. These data have enabled the inventor to predict the most potent GnRH II analog structure for the GnRH II receptor in the immune system, and assist in the design of even more potent analogs for the GnRH receptor.

In these studies, GnRH receptors have been purified from human immune system tissue after ethanol precipitation and extraction of GnRH in the supernatant. The binding affinity for the receptor free and containing supernatants were compared for each GnRH II or analog have been compared. Each study has been done using two different human immune system tissues.

### Example XI

### Activity of GnRH II or its Analogs on Immune System Tissues

Tissues of the immune system have been examined for the ability of their cells in vitro to respond to GnRH II in culture medium. The media from cell cultures of immune system tissues have been examined for the release of cytokine into the medium after incubation with and without GnRH II analog.

Cell cultures of human leukocytes tissues have been prepared. These cells have been cultured in the presence and absence of GnRH II and its analogs and GnRH I and its analogs at varying doses. The release of cytokines into the medium have been determined and compared for each form of GnRH studied. GnRH II and its analogs had greater activity on immune systems cytokines and GnRH II analog was the most active.

Applicant studied the effect of cytokines produced by T cells. Applicant found no effects of GnRH II, or Applicant's GnRH II analog on Interferon γ (INFγ), IL-4, IL-8 and IL-10 on the low production of these cytokines by any of these peptides using this system. Concentrations of 2×10⁻⁹ to 2×10⁻⁷ M at 1, 3 and 20 hours were studied.

Applicant also studied cytokine produced primarily by B cells and macrophages. Applicant observed that GnRH II analog inhibited Interleukin 12 (IL-12) after even one hour of treatment, which was still observed at 3 and 20 hours. The natural isoform of GnRH II also effected an inhibition at 3 hours while GnRH I increased IL-12 followed by a decrease at 20 hours using high dose of GnRH I. This is consistent with opposing activities followed by down regulation of the receptors with chronic high concentrations of the ligand.

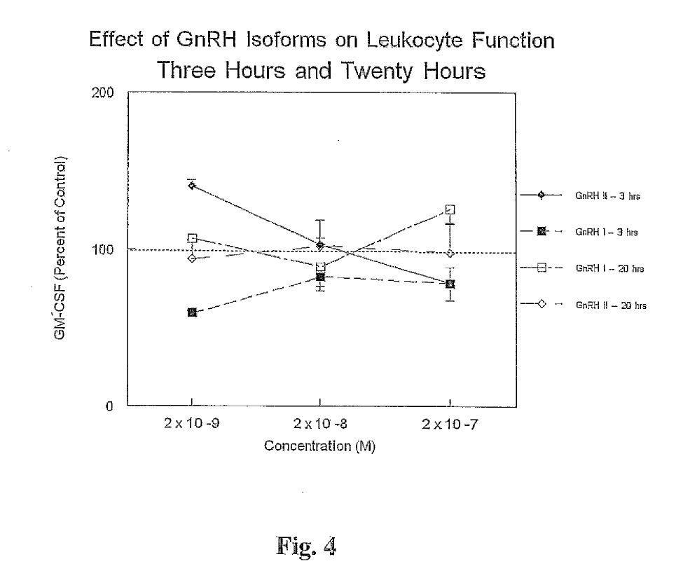

Applicant also demonstrated that GnRH II at low dose is a potent stimulant of granulocyte macrophage colony stimulating factor, GM-CSF, while at high dose inhibits this cytokine as expected with down-regulation or the GnRH II receptor. The activity is observed at 3 hrs but not at 20 hours due to the limited stability of GnRH in biological fluids. FIG. 4 shows the effect of GnRH isoforms I and II on leukocyte function, specifically granulocyte/macrophage colony stimulating factors (GM-CSF) release from human leukocytes, at three hours and twenty hours. Using Applicant's GnRH II analog an inhibition of GM-CSF was clearly apparent after 20 hours of exposure. Monocyte production of IL-10 was shown to be affected agonistically. In FIG. 7, after twenty four hours of incubation with applicant's analog, production of IL-10 increased. GM-CSF was also increased by this analog.

Applicant also demonstrated that Applicant's analog is an antagonist of GnRH II activity of PGE and TNFα. See FIGS. 6A and 6B. Monocyte production of PGE was shown to be affected antagonistically by Applicant's analog. PGE was measured after three hours of exposure to the analog in a dose range of 10⁻¹⁰ to 10⁻⁷M (signified by the triangles in FIG. 6A) and three hours of exposure to a combination Applicant's analog and GnRH II at a dose of 10⁻⁷M (signified by the open circles in FIG. 6A). Applicant's analog induced PGE production at 10⁻⁸ and 10⁻⁷M. However, addition of GnRH II reversed the PGE activity.

Similarly, monocyte production TNFα was shown to be affected antagonistically by Applicant's analog. TNFα was measured after three hours of exposure to the analog in a dose range of 10⁻¹° to 10⁻⁷M (signified by the solid squares in FIG. 6B) and three hours of exposure to a combination Applicant's analog and GnRH II at a dose of 10⁻⁷M (signified by the open squares in FIG. 6B). Applicant's analog induced TNFα production at 10⁻⁸ and 10⁻⁷M. However, addition of GnRH II reversed the TNFα activity.

Applicant also demonstrated that monocyte production of IL-10 was shown to be affected agonistically. In FIG. 7, “nIIA” represents the embodiment of Applicant's analogs D-Asn(6)-GnRH II aza-Gly-NH₂. After twenty four hours of incubation with applicant's analog, production of IL-10 increased. GM-CSF was also increased by this analog.

While the compositions and methods of this invention have been described in terms of preferred embodiments, it will be apparent to those of skill in the are that variations may be applied to the composition, methods and in the steps or in the sequence of steps of the method described herein without departing from the concept, spirit and scope of the invention.

I claim:

1. A gonadotropin releasing hormone II analog having the sequence as defined in SEQ ID NO: 3, p-Glu-His-Trp-Ser-His-Xaa-Trp-Tyr-Pro-Xaa, said analog comprising a D-amino acid substitution at position 6 and an aza-Gly-amide substitution, or other amino acid-amide substitution at position 10;
wherein said analog resists degradation by post-proline peptidases and endopeptidases in immune tissues and other tissues where immune cells or immune tissues are found;
wherein said analog binds with higher affinity to gonadotropin releasing hormone receptors in the immune system than native gonadotropin releasing hormone I or native gonadotropin releasing hormone II; and
wherein said analog is a non-chimaeric analog of gonadotropin releasing hormone II.

2. The gonadotropin releasing hormone II analog as recited in claim 1 wherein said D-amino acid is selected from the group consisting of D-Arg, D-Leu, D-Asn, D-tBu-Ser or D-Trp.

3. The gonadotropin releasing hormone II analog as recited in claim 1 wherein said sequence of said analog is p-Glu-His-Trp-Ser-His-D-Arg-Trp-Tyr-Pro-aza-Gly-NH₂.

4. The gonadotropin releasing hormone II analog as recited in claim 1 wherein said sequence of said analog is p-Glu-His-Trp-Ser-His-D-Asn-Trp-Tyr-Pro-aza-Gly-NH₂.

5. The gonadotropin releasing hormone II analog as recited in claim 1 wherein said sequence of said analog is p-Glu-His-Trp-Ser-His-D-Leu-Trp-Tyr-Pro-aza-Gly-NH_{2.}

6. The gonadotropin releasing hormone II analog as recited in claim 1 wherein said analog acts as an agonist or antagonist in said immune system, said immune tissues and said other tissues where immune cells or immune tissues are found.

7. The gonadotropin releasing hormone II analog as recited in claim 3 wherein said analog is an antagonist in said immune system, said immune tissues and said other tissues where immune cells or immune tissues are found.

8. The gonadotropin releasing hormone II analog as recited in claim 4 wherein said analog is an agonist in said immune system, said immune tissues and said other tissues where immune cells or immune tissues are found.

9. The gonadotropin releasing hormone II analog as recited in claim 5 wherein said analog is an agonist in said immune system, said immune tissues and said other tissues where immune cells or immune tissues are found.

10. A method of monitoring treatment of immunological disorders and diseases in a human patient comprising:
first administering to said patient's the immune system, immune tissues or other tissues where immune cells or immune tissues are found, a gonadotropin releasing hormone II analog having the sequence as defined in SEQ ID NO: 3, p-Glu-His-Trp-Ser-His-Xaa-Trp-Tyr-Pro-Xaa, wherein said analog has a D-amino acid substitution at position 6 and an aza-Gly-amide substitution, or other amino acid-amide substitution at position 10;
second administering a gonadotropin releasing hormone antibody into said immune system of said patient; and
determining an amount of said analog not bound to a GnRH receptor in said immune system.

11. The method of monitoring treatment of immunological disorders and immunological diseases as recited in claim 10 wherein said D-amino acid is selected from the group consisting of D-Arg, D-Leu, D-Asn, D-tBu-Ser or D-Trp.

12. The method of monitoring treatment of immunological disorders and immunological diseases as recited in claim 10 wherein said sequence of said analog is p-Glu-His-Trp-Ser-His-D-Arg-Trp-Tyr-Pro-aza-Gly-NH₂ and acts as an antagonist in said immune system, said immune tissues and said other tissues where immune cells or immune tissues are found.

13. The method of monitoring treatment of immunological disorders and immunological diseases as recited in claim 10 wherein said sequence of said analog is p-Glu-His-Trp-Ser-His-D-Asn-Trp-Tyr-Pro-aza-Gly-NH₂ and acts as an agonist in said immune system, said immune tissues and said other tissues where immune cells or immune tissues are found.

14. The method of monitoring treatment of immunological disorders and immunological diseases as recited in claim 10 wherein said sequence of said analog is p-Glu-His-Trp-Ser-His-D-Leu-Trp-Tyr-Pro-aza-Gly-NH₂ and acts as an agonist in said immune system, said immune tissues and said other tissues where immune cells or immune tissues are found.

15. The method of monitoring treatment of immunological disorders and immunological diseases as recited in claim 10 wherein said antibody used in said second administering step is an antibody to said analog, and said determining step comprises measuring an amount of antibody bound to said analog to determine an amount of said analog not bound to said GnRH receptor.

16. The method of monitoring treatment of immunological disorders and immunological diseases as recited in claim 10 wherein said antibody used in said second administering step is an antibody to said GnRH receptor, and said determining step comprises measuring an amount of antibody bound to said GnRH receptor to determine an amount of said analog not bound to said GnRH receptor.

17. The method of monitoring treatment of immunological disorders and immunological diseases as recited in claim 10 wherein said first administering step comprises administering said analog through a pharmaceutical formulation by injection, subdermal pellet, nasal spray, orally, subcutaneously, intrapulmonary, transmucosally, intraperitoneallly, sublingually, or by intrathecal routes.

18. A method of treating immunological disorders and diseases in a human patient comprising administering to said patient's immune system, immune tissues or other tissues where immune cells or immune tissues are found, a gonadotropin releasing hormone II analog having the sequence as defined in SEQ ID NO: 3, p-Glu-His-Trp-Ser-His-Xaa-Trp-Tyr-Pro-Xaa, wherein said analog has a D-amino acid substitution at position 6 and an aza-Gly-amide substitution, or other amino acid-amide substitution at position 10;
wherein said analog binds with high affinity to GnRH receptors within said immune system, said immune tissues and said other tissues where immune cells or immune tissues are found.

19. The method of treating immunological disorders and diseases as recited in claim 18 wherein said sequence of said analog is p-Glu-His-Trp-Ser-His-D-Arg-Trp-Tyr-Pro-aza-Gly-NH₂ and acts as an antagonist in said immune system, said immune tissues and said other tissues where immune cells or immune tissues are found.

20. The method of treating immunological disorders and diseases as recited in claim 18 wherein said sequence of said analog is p-Glu-His-Trp-Ser-His-D-Asn-Trp-Tyr-Pro-aza-Gly-NH₂ and acts as an agonist in said immune system, said immune tissues and said other tissues where immune cells or immune tissues are found.

21. The method of treating immunological disorders and diseases as recited in claim 18 wherein said sequence of said analog is p-Glu-His-Trp-Ser-His-D-Leu-Trp-Tyr-Pro-aza-Gly-NH₂ and acts as an agonist in said immune system, said immune tissues and said other tissues where immune cells or immune tissues are found.

22. The method of treating immunological disorders and diseases as recited in claim 18 wherein said analog is administered into said patient's immune system through a pharmaceutical formulation by injection, subdermal pellet, nasal spray, orally, subcutaneously, intrapulmonary, transmucosally, intraperitoneallly, sublingually, or by intrathecal routes.

23. A method of diagnosing immunological disorders and diseases in a human patient comprising:
administering an antibody to said patient's immune system, immune tissues or other tissues where immune cells or immune tissues are found to bind to a gonadotropin releasing hormone II substance; and
measuring a binding level of said antibody to said gonadotropin releasing hormone II substance to determine and detect said immunological disorders and diseases.

24. The method of diagnosing immunological disorders and diseases as recited in claim 23 wherein said antibody is an antibody to gonadotropin releasing hormone II, and said gonadotropin releasing hormone II substance is said gonadotropin releasing hormone II as defined as defined by SEQ. ID. NO.: 2, p-Glu-His-Trp-Ser-His-Gly-Trp-Tyr-Pro-Gly.

25. The method of diagnosing immunological disorders and diseases as recited in claim 23 wherein said antibody is an antibody to gonadotropin releasing hormone II receptors, and said gonadotropin releasing hormone II substance is said gonadotropin releasing hormone II receptor.

26. The method of diagnosing immunological disorders and diseases as recited in claim 23 wherein said antibody is an antibody to gonadotropin releasing hormone II analogs, and said gonadotropin releasing hormone II substance is a gonadotropin releasing hormone II analog having the sequence as defined in SEQ ID NO: 3, p-Glu-His-Trp-Ser-His-Xaa-Trp-Tyr-Pro-Xaa, wherein said analog has a D-amino acid substitution at position 6 and an aza-Gly-amide substitution, or other amino acid-amide substitution at position 10.

27. The method of diagnosing immunological disorders and diseases as recited in claim 26 wherein said D-amino acid is selected from the group consisting of D-Arg, D-Len, D-Asn, D-tBu-Ser or D-Trp.

28. The method of diagnosing immunological disorders and diseases as recited in claim 26 wherein said sequence of said analog is p-Glu-His-Trp-Ser-His-D-Arg-Trp-Tyr-Pro-aza-Gly-NH_{2.}

29. The method of diagnosing immunological disorders and diseases as recited in claim 26 wherein said sequence of said analog is p-Glu-His-Trp-Ser-His-D-Asn-Trp-Tyr-Pro-aza-Gly-NH_{2.}

30. The method of diagnosing immunological disorders and diseases as recited in claim 26 wherein said sequence of said analog is p-Glu-His-Trp-Ser-His-D-Leu-Trp-Tyr-Pro-aza-Gly-NH_{2.}


SEQUENCE LISTING (3 sequences)

SEQ ID NO: 1 - protein, 10 aa, Gallus gallus
  mat_peptide Unknown: Chicken I GnRH.
EHWSYGLQPG

SEQ ID NO: 2 - protein, 10 aa, Gallus gallus
  mat_peptide Unknown: Chicken II GnRH.
EHWSHGWYPG

SEQ ID NO: 3 - protein, 10 aa, Unknown
  Chicken II GnRH Analog. MOD_RES Glu at position 1 is pyroglutamic acid.
EHWSHXWYPX